(19) United States
(12) Patent Application Publication
(10) Pub. No.: US 2009/0149562 A1
(43) Pub. Date: Jun. 11, 2009

(54) PURIFICATION OF A DISTILLED BOTTOM PRODUCT CONTAINING (METH)ACRYLIC ACID BY MEANS OF A CRYSTALLIZATION PROCESS

(76) Inventors: Stefan Nordhoff, Recklinghausen (DE); Thomas Rathke, Mandeville, LA (US); Torsten Balduf, Houston, TX (US); Axel Kobus, Bochum (DE); Dennis Thong Yu-Chiang, Marl (DE); Jorg Leistner, Dortmund (DE)

Correspondence Address: SMITH MOORE LEATHERWOOD LLP, P.O. BOX 21927, GREENSBORO, NC, 27420, US

(21) Appl. No.: 11/575,157

(22) PCT Filed: Sep. 13, 2005
(86) PCT No.: PCT/EP05/09835

§ 371 Date: Aug. 19, 2008

(30) Foreign Application Priority Data
Sep. 13, 2004 (DE) 10 2004 044 638.5

(51) Int. Cl. C07C 51/42; B01J 19/00; C08G 63/00

(52) U.S. Cl. 521/149; 562/600; 422/187; 528/271; 526/317.1; 521/182

(57) ABSTRACT

A process for the preparation of (meth)acrylic acid is disclosed that includes:
(a) contacting a product gas containing (meth)acrylic acid obtained from a gas phase oxidation with an aqueous phase in a quenching unit to obtain an aqueous quenched phase,
(b) distilling the quenched phase in a distillation unit to obtain a bottom product and a top product, and
(c) crystallizing at least the bottom product in a crystallization unit to obtain pure (meth)acrylic acid.
   An organic separating agent can be added after the quenching unit, such as at the latest in the crystallization unit. Also disclosed are a device for the preparation of (meth)acrylic acid, a process for the preparation of a polymer from the (meth)acrylic acid, a device for the preparation of the polymer, a polymer obtainable by this process, and the use of the (meth)acrylic acid or the polymer in a chemical product, such as fibers or shaped articles.

This application is a national stage application under 35 U.S.C. 371 of international application No. PCT/EP2005/009835 filed 13 Sep. 2005, and claims priority to German Application No. DE 10 2004 044 638.5 filed 13 Sep. 2004, the disclosure of which is expressly incorporated herein by reference.

The invention relates to a process for the preparation of (meth)acrylic acid, a device for the preparation of (meth)acrylic acid, a process for the preparation of a polymer from a (meth)acrylic acid produced by the above process, a device for the preparation of this polymer, a polymer produced by this process, and the use of the (meth)acrylic acid or the polymer in chemical products, such as fibers or shaped articles.

In the present context, (meth)acrylic acid is understood as meaning methacrylic acid and acrylic acid.

(Meth)acrylic acid, and in particular acrylic acid, is a monomer which is currently used in many polymers. In particular, acrylic acid is used in the preparation of polymers which are employed for water treatment, for example as flocculating agents, or which are incorporated as superabsorbent polymers into hygiene articles, in particular diapers. When used both in the field of water treatment and in the field of hygiene articles, very high purity requirements are expected of the polymers employed there. This is the case in particular with hygiene articles, such as diapers and in which the polymer comes into contact with skin, directly or indirectly. This skin is very sensitive to contaminants if they are irritating or toxic.

It is generally known that acrylic acid and methacrylic acid can be prepared by heterogeneous catalyzed gas phase oxidation of unsaturated hydrocarbons with oxygen on catalysts which are in general in the solid state of aggregation, at temperatures of between 200 and 400° C. See DE OS 19 62 431, DE OS 29 43 707 and DE 108 38 845 A1.

It is also known from WO 03/051809 A1 to bring a product gas containing (meth)acrylic acid obtained from the gas phase oxidation of propylene into contact with an aqueous phase to give an aqueous quenched phase. In such a quenched phase, the (meth)acrylic acid is still accompanied by various other reaction products, which are regarded as impurities, and by water as an absorption agent. This makes it necessary to feed the quenched phase obtained above to at least one further purification process. This purification process is often a distillation. Any distillation method with which persons skilled in the art are familiar and that seems suitable can be used. However, as a rule, the (meth)acrylic acid contained in the quenched phase is not successfully purified via distillation efficiently enough so that (meth)acrylic acid and impurities on water are obtained. Rather, in addition to the desired purification, a noticeable amount of (meth)acrylic acid remains at the bottom of a column employed for distillative working up of the quenched phase. It would, however, be unprofitable and environmentally unsound to discard this bottom product without further purification.

Various set-ups are known from the prior art for purification of (meth)acrylic acid. For example, DE 198 38 845 A1 discloses a process for the purification of acrylic acid which can be crystallized out directly from a mixture with solvents, wherein the crystals can be in the form of, for example, a crystal suspension. This specification discloses the purification of contaminated acrylic acid which contains comparatively little water or is even anhydrous.

Japanese Patent Specification JP 32417/1970 discloses a process for the purification of acrylic acid, wherein a product gas obtained by gas phase oxidation is first absorbed with an absorption agent, such as water, in a quenching unit, and the quenched phase is fed to an extraction column in which a water-containing extraction phase is produced, which is fed to a distillation unit for removal of the water, the bottom product of this distillation column being subjected to crystallization. This process has disadvantages since a further extraction step is also inserted in between the absorption in the quenched phase and the distillation for the dewatering.

EP 1 116 709 A1 discloses a process for the purification of crude acrylic acid which is obtained by distillation of a quenched phase originating from a quenching unit, wherein this crude acrylic acid essentially contains none of the solvents of the bottom product of the column, and thus essentially contains no water. One of the disadvantages of the process described in this publication is that this process is carried out with a very high separation effect, so that scarcely any separating agent remains in the bottom product, Because of the increased exposure to heat, this leads, inter alia, to an increased formation of polymer in the column.

In general, the present invention is based on the object of overcoming the disadvantages arising from the prior art.

One object of the present invention is to provide a process for the purification of (meth)acrylic acid which can be operated in a both environment-friendly and economical manner.

Another object of the present invention is to provide a process for the purification of (meth)acrylic acid, in which the (meth)acrylic acid, which is known to be a reactive monomer, can be purified as carefully as possible.

A further object of the present invention is to provide a process for the preparation of (meth)acrylic acid, and for the purification thereof, that can be carried out with as few steps as possible.

The objects described above are achieved by the present invention.

The invention thus relates to a continuous process for the preparation of (meth)acrylic acid, wherein
(a) a product gas containing (meth)acrylic acid and obtained from a gas phase oxidation is brought into contact with an aqueous phase in a quenching unit to give an aqueous quenched phase,
(b) the quenched phase is subjected to a distillation in a distillation unit to give a bottom product and a top product,
(c) at least the bottom product is subjected in a crystallization unit to give pure (meth)acrylic acid,
   wherein an organic separating agent is added after the quenching unit and, in one embodiment, at the latest in the crystallization unit.

According to one embodiment of the present invention, the quenched phase can contain, as quenched phase components,
Q1 from about 45 to 85 wt. %, in another aspect of an embodiment from about 50 to 80 wt. %, and in a further aspect of an embodiment from about 55 to 65 wt. %, of (meth)acrylic acid,
Q2 at least 14.9 wt. %, in another aspect of an embodiment at least 19 wt. %, and in a further aspect of an embodiment at least 30 wt. %, of water and
Q3 at least 0.1 wt. %, in another aspect of an embodiment from about 1 to 10 wt. %, in a further aspect of an embodiment from about 2 to 5 wt. %, of an impurity other than Q1 and Q2, wherein each amount stated in each case is based on the total weight of the quenched phase,
and wherein the sum of the wt. % stated for the quenched phase components Q1 to Q3 is 100.

In process step (b), the quenched phase can be fed in principle to any distillation device known to be suitable for purification of the quenched phase. According to one embodiment of the present invention, distillation columns which are suitable for azeotropic distillation can be utilized. These can be
tray columns, in one aspect of an embodiment containing vapor deflection hoods, such as, for example, bubble trays, tunnel trays, ripple trays or S trays, wherein the trays have simple openings for passage of vapor (perforated trays or grid trays in the absence of vapor deflection or jet trays, expanded metal trays or centrifugal trays if a vapor deflection is present), flexible openings for passage (valve trays with disc, blast or lever valves) or cascade-like openings for passage of vapor;
packed columns containing bulk packing, such as hollow cylinder packing, such as, for example, RASCHIG rings with and without inserts (super-rings), INTOS rings, PALL rings, wire mesh rings, expanded metal rings, coiled rings, WILSON spiral rings or PRYM rings, spool-like packing, such as, for example, Haltmeier rolls, saddle-shaped packing, such as, for example, BERL saddles, INTALOX saddles or wire mesh saddles, cross-shaped packing, such as, for example, geminate bodies, propeller bodies or star-shaped bodies, box-like packing, such as, for example, HELI-PAK bodies, OCTA-PAK bodies, and spherical packing, such as, for example, ENVI-PAK bodies, or packed column containing packing inserts (packing columns), such as braided wire inserts (STEDMAN BODIES, SPRAY-PAL bodies) or coiled wire inserts, glass inserts, such as, for example, borosilicate glass inserts (Durapack), stacked sheet inserts, such as, for example, trickle sheet inserts (trickle sheet columns), expanded metal inserts (PANA-PAK, PERFORM-GRID) and vertical tube inserts, such as, for example, tube bundle inserts (KUHN columns),
rotary columns, for example rotary columns according to E. KIRSCHBAUM, and
horizontal columns.

In process step (b), after the distillation of the quenched phase a bottom product is obtained, and in one embodiment the bottom product can be a single phase. According to the present invention, the term “single phase” means that a sample does not undergo phase separation into two or more phases for at least one hour at 20° C. A single-phase bottom product can be pumped more easily, and deposits form less readily in the lines in which the bottom product is introduced.

In another embodiment of the present invention, the bottom product can contain
S1 at least 80 wt. %, in another aspect of an embodiment at least 85 wt. %, and in a further aspect of an embodiment from about 89.99 to 95 wt. %, of (meth)acrylic acid,
S2 at most 5 wt. %, in another aspect of an embodiment at most 1 wt. %, and in a further aspect of an embodiment from about 0.01 to 1 wt. %, of water,
S3 at least 1 wt. %, in another aspect of an embodiment at least 2.5 wt. %, in a further aspect of an embodiment at least 5 wt. %, in yet another aspect of an embodiment at least 7.5 wt. %, and in yet another aspect of an embodiment from about 5 to 15 wt. %, of separating agent and
S4 at least 0.01 wt. %, in another aspect of an embodiment at least 0.05 wt. %, and in a further aspect of an embodiment from about 0.1 to 5 wt. %, of impurities other than S1 to S3, wherein each amount stated in each case is based on the total weight of the bottom product.
   and wherein the sum of the wt. % stated for the bottom product components S1 to S4 is 100. In one embodiment of the present invention, purification of such bottom products results in an increase in the profitability of the process for the preparation of (meth)acrylic acid.

The bottom product obtained in process step (b) is then subjected to process step (c), in which pure (meth)acrylic acid is obtained by means of a crystallization, which can be a suspension crystallization or a layer crystallization.

According to one embodiment of the process of the present invention, the pure (meth)acrylic acid contains at least 97.5, in another aspect of an embodiment at least 98.0, in a further aspect of an embodiment at least 99.0, and in yet another aspect of an embodiment 99.5 wt. %, of (meth)acrylic acid. Such units of (meth)acrylic acid are useful when a polymer prepared from the (meth)acrylic acid is used in the hygiene field, in the field of wound dressings and bandages, or in other medical fields, such as medicament formulation or medical technology.

In principle, according to the invention, all the substances known to persons skilled in the art can be used as the organic separating agent employed after the quenching unit and at the latest in the crystallization unit, and in one embodiment the separating agent can be in the form of a liquid at 20° C. Organic separating agents in particular have proved suitable in the process according to the invention. These include hydrocarbons, halogenated hydrocarbons, carbonyl compounds, alcohols, carboxylic acids, carboxylic acid esters, ethers, polyethers and an organic sulphur or phosphorus compound.

Separating agents which can be utilized in the present invention include
a hydrocarbon, such as, for example, n-hexane, n-heptane, dimethylcyclohexane, ethylcyclohexane, aromatics with an alkyl group, such as, for example, toluene, xylene or ethylbenzene, halogenated hydrocarbons, in particular halogenated aromatics, such as, for example, chlorobenzene, or mixtures of the above-mentioned hydrocarbons,
a carbonyl compound, such as, for example, acetone, acetaldehyde, diethyl ketone, diisopropyl ketone, methyl propyl ketone, methyl isobutyl ketone, methyl t-butyl ketone, n-nonanone or mixtures of these carbonyl compounds,
an alcohol or a polyol, such as, for example, methanol, n-propanol, iso-propanol, n-butanol, iso-butanol, tert-butanol, 1-decanol, glycerol or mixtures of these alcohols,
a carboxylic acid or a carboxylic acid ester, such as, for example, formic acid, acetic acid, n-propyl acetate, n-butyl acetate, ethyl acrylate, methyl methacrylate, ethyl methacrylate, vinyl acrylate, n-propyl acrylate, allyl acetate, isopropenyl acetate, vinyl propionate, propyl propionate, methyl crotonate, methyl valerate, ethyl butyrate or mixtures of these carboxylic acids or carboxylic acid esters,
an ether or a polyether, such as, for example, dimethyl ether, diethyl ether, ethyl methyl ether, dipropyl ether, methyl propyl ether, ethyl propyl ether, methyl propyl ether, ethyl methyl ether, dibutyl ether, ethylene glycol, propylene glycol, diethylene glycol dimethyl ether, diethylene glycol diethyl ether, triethylene glycol dimethyl ether and mixtures of these ethers or polyethers,
an organic sulphur or phosphorus compound, such as, for example, methanesulphonic acid, dioctylmonohexaphosphine (obtainable under the trade name Cyanex 923® from Cytec), trioctylphosphone oxide or mixtures of these compounds,
a solvent mixture containing a solvent A chosen from the group consisting of heptane, dimethylcyclohexane, ethylcyclohexane, toluene, benzene, ethylbenzene, chlorobenzene, xylene or a mixture of these solvents and a solvent B chosen from the group consisting of diethyl ketone, diisopropyl ketone, methyl propyl ketone, methyl isobutyl ketone, ethyl acrylate, methyl methacrylate, ethyl methacrylate, vinyl acrylate, n-propyl acrylate, allyl acetate, isopropenyl acetate, vinyl propionate, propyl propionate, methyl crotonate, methyl valerate, ethyl butyrate, dibutyl ether and mixtures of these solvents.

Separating agents which can be utilized in embodiments of the present invention are aromatics, and in an aspect of an embodiment are aromatics containing at least one alkyl group. Among these compounds, toluene, o-xylene, m-xylene, p-xylene, mesitylene, 1,4-trimethylbenzene, ethylbenzene or p-cymene or mixtures of at least two of these compounds are utilized in further aspects of an embodiment, and toluene is utilized as the separating agent in yet another aspect of an embodiment.

In one embodiment of the process according to the present invention, this process is carried out continuously. This means that the quenched phase obtained in process step (a) is fed continuously to the distillation device and the bottom product obtained in the distillation unit is removed and fed to the crystallization unit in a continuous manner. In this context, the separating agent is added after the quenching unit and at the latest in the crystallization unit.

In another embodiment of the continuous process of the present invention, the separating agent retained during the purification of the (meth)acrylic acid by means of crystallization, in one aspect of an embodiment by means of suspension crystallization or by means of layer crystallization, can be at least partly added again after the quenching unit and at the latest to the crystallization. A very efficient use of the separating agent is ensured by this procedure. In one embodiment, the separating agent retained during the crystallization can be recycled by recycling the mother liquor which is retained after separating off the (meth)acrylic acid crystals in the crystallization unit and has a high content of separating agent into the distillation unit. In another embodiment of the process of the present invention the recycling of the mother liquor which has a high content of separating agent can be carried out such that this is first introduced through a separating unit (separator), which is, for example, a crystallization unit, in one aspect of an embodiment a layer crystallizer or a suspension crystallization unit comprising a crystal suspension generator and a separating device, in another aspect of an embodiment a washing column, or a centrifuge, a filter or another separating device suitable for separating off impurities, in order first to separate off impurities still present, in particular high-boiling components or low-boiling components, such as, for example, acetic acid. The mother liquor which is purified in this manner and has a high content of separating agent is then recycled into the process in the manner and method described above. In one embodiment of the process according to the present invention, high-boiling components are separated off from the mother liquor by means of a separating unit, and in another embodiment low-boiling components, such as, for example, acetic acid, are separated off. The use of at least two separation units arranged in series is also conceivable, so that first high-boiling components and then low-boiling components (or vice versa) can be separated off.

In connection with the recovery of the separating agent, it has furthermore proved appropriate according to one embodiment of the present invention for the top product to be at least partly separated into a phase having a high content of separating agent and an aqueous phase having a low content of separating agent, this separation taking place in a separation device in another embodiment. This measure also contributes towards increasing the efficiency of the process according to the invention. In another embodiment the top product originating from the distillation has a higher temperature on leaving the distillation unit than during its further treatment in the separation device. In a further embodiment, the phase obtained in the separation device, which has a high content of separating agent, is fed to the distillation unit and the aqueous phase having a low content of separating agent is at least partly fed to the quenching unit. In one aspect of an embodiment the separating agent can be at least partly added after the quenching unit and at the latest in the crystallization unit.

The amount of separating agent employed in the process according to the invention relative to the amount of quenching phase depends on the extent to which separating agent which has already been brought into contact with the quenched phase is recycled back into the process in accordance with the above statements via the recycling of the mother liquor retained in the crystallization unit or via the recycling of the phase obtained in the separation device which has a high content of separating agent. A loss in separating agent can thus be caused, for example, by separating off secondary components. In this context, the person skilled in the art will be able to determine the amount of separating agent required for a satisfactory purification of the (meth)acrylic acid contained in the quenched phase as a function of the given process parameters (extent of recycling of the separating agent, composition of the quenched phase, nature of the distillation and crystallization processes used, reaction temperature, reaction pressure and the like) by simple routine experiments,

In one embodiment of the process according to the invention, the separating agent is employed in an amount from about 10 to 90 wt. %, in another aspect of an embodiment in an amount from about 30 to 80 wt. %, in a further aspect of an embodiment from about 50 to 70 wt. %, based on the total weight of separating agent and of the quenched phase employed. In this context, the weight ratio of freshly employed (i.e., not yet brought into contact with the quenched phase or components of the quenched phase) separating agent to recycled separating agent (via the mother liquor obtained in the crystallization unit and via the phase obtained in the separation device which has a high content of separating agent) is, in one aspect of an embodiment from about 1:2,000 to 1:10, in another embodiment from about 1:1,000 to 1:20, and in a further embodiment of from about 1:200 to 1:100.

As described above, the organic separating agent is employed after the quenching unit and in one embodiment at the latest in the crystallization unit. In one embodiment, the organic separating agent can be added
into the feed line with which the quenched phase is led into the distillation unit,
into the distillation unit, and in one aspect of an embodiment into the upper region of the distillation unit,
into the feed line with which the bottom product is led into the crystallization unit,
into the crystallizer of the crystallization unit,
into the feed line with which, in the case of a suspension crystallization unit, the crystal suspension is led into the separating device, in one aspect of an embodiment the washing column,
into the separating device, in one aspect of the embodiment the washing column,
into the feed line with which the mother liquor retained in the crystallization unit is at least partly recycled into the distillation unit,
in the case where a separation device is employed, into the feed line with which the phase having a high content of separating agent is recycled into the distillation unit, into the feed line with which the top product of the distillation unit is passed into the separation device, or into the separation device itself.

In one embodiment of the process of the present invention, in process step c) the bottom product which has been obtained in process step b) is fed to a suspension crystallization unit comprising a crystallizer and a separating device, in one aspect of an embodiment a washing column, the mother liquor obtained in the suspension crystallization unit being recycled back into the distillation unit. In another embodiment of the process according to the invention, in process step c) the bottom product which has been obtained in process step b) is fed to a layer crystallizer, the mother liquor obtained n the layer crystallizer being recycled back into the distillation unit.

The invention furthermore relates to a device for the preparation of (meth)acrylic acid, comprising, connected to one another in series by fluid-carrying lines,
a reactor unit,
a quenching unit,
a distillation unit,
a crystallization unit comprising a crystallizer, in one embodiment being a layer crystallizer or a suspension crystallization unit, wherein, in the case of a suspension crystallization unit, this also comprises, in addition to the crystallizer, a separating device, in one aspect of an embodiment being a washing column. In the case of layer crystallization, a separating device is usually not necessary.

According to the invention, fluid-carrying is understood as meaning that the lines, which in one embodiment are pipelines, are constructed and configured such that these can carry gases or liquids or supercritical fluids or solids as a slurry in liquids or at least two of these.

In one embodiment of the device according to the invention, crystallizer can be connected to a region of the lower half, in one aspect of an embodiment to a region of the lower half, and in another aspect of an embodiment to a region of the lower third, and in a further aspect of an embodiment to a region of the lower quarter of the distillation unit. This measure renders it possible for the bottom product to be removed with as few problems as possible. Furthermore, in an embodiment of the device according to the present invention, in one aspect of an embodiment there can be a separating agent feed from the layer crystallizer or the suspension crystallization unit, including one from the washing column of the suspension crystallization unit, that opens into a region of the upper half, including one that opens into a region of the upper half, and in one aspect of an embodiment into a region of the upper third, and in another aspect of the embodiment one that opens into a region of the upper quarter of the distillation unit. The mother liquid which is retained in the crystallization unit and has a high content of separating agent can be recycled into the process via this line.

Layer crystallizers which can be employed are those dynamic or static layer crystallizers disclosed as static or dynamic layer crystallizers in EP 0616 998 A1.

Any device suitable to persons skilled in the art in providing crystal suspensions can be employed as the crystallizer in the suspension crystallization unit. One embodiment includes crystallizers which allow the process according to the invention to be carried out continuously. A stirred tank crystallizer, a scraped film crystallizer, a cooled disc crystallizer, a crystallizing screw, a drum crystallizer or the like can advantageously be arranged in the device according to the invention as the crystallizer. This crystallizer in one embodiment can be the cooled disc crystallizer or the scraped film cooler (see Poschmann, Suspensionskristallisation organischer Schmelzen und Nachbehandlung der Kristalle durch Schwitzen oder Waschen [Suspension Crystallization of Organic Melts and After-treatment of the Crystals by Exudation or Washing], University of Bremen, Shaker Verlag, Aachen 1996). In one embodiment, the device according to the present invention can comprise a scraped film cooler as the crystallizer.

In one embodiment of the present invention, the suspension crystallization unit has a separating device for separating off the (meth)acrylic acid crystals, and in one aspect of the embodiment this separating device is a washing column. In another aspect of an embodiment, a hydraulic washing column can be employed, wherein the suspension can be introduced in the upper part of the column. The mother liquor is stripped off from the column via a filter, as a result of which a densely packed crystal bed forms. The crystal bed and the mother liquor flow in the direction of the base of the washing column. At the base of the washing column is a moving, and in one aspect of an embodiment rotating, scraping device or a scraper, which produces a suspension again from the densely packed crystal bed and the wash melt introduced at the lower part of the washing column. This suspension can be pumped through a melter, in one aspect of an embodiment a heat exchanger, and melted. A part of the melt can serve e.g., as the wash melt. This is then pumped back into the column, and in one aspect of an embodiment washes out the crystal bed migrating in the opposite direction, i.e. the crystallized (meth)acrylic acid is washed by the recycled (meth)acrylic acid in counter-current. The wash melt effects washing of the crystals and also at least partly crystallizes out on the crystals. The crystallization enthalpy that is released heats the crystal bed in the washing region of the column. As a result, a purification effect analogous to exudation of the crystals is achieved. A purification is thus effected by the washing of the surface of the (meth)acrylic acid crystals with molten—and therefore already purified—(meth)acrylic acid, and a healing or exudation of impurities is achieved by the crystallization of the molten, purified (meth)acrylic acid on the (meth)acrylic acid crystals already present. This allows highly pure preparation of (meth)acrylic acid. The washing column thus serves for solid-liquid separation and for carrying out a displacement washing, the displacement washing being carried out without loss of washing liquid.

In another embodiment of the present invention, the separating off of the (meth)acrylic acid by crystallization of the (meth)acrylic acid in the layer crystallizer or suspension crystallization unit can be carried out in one or more stages. In a two-stage crystallization, for example, the mother liquor retained in the washing column or in the layer crystallizer after the (meth)acrylic acid crystals have been separated off is fed to a second crystallization unit. The second crystallization unit can be a layer crystallizer or a suspension crystallization unit. In the second crystallization unit, (meth)acrylic acid crystals are separated off to give a further mother liquor.

A distillation and quenching unit which can be used according to the present invention is disclosed in DE 198 38 845 A1 and WO 03/051809 A1, in WO 03/051809 A1. These references are herein specifically incorporated by reference.

Furthermore, in an embodiment of the device according to the present invention, a separation device can be connected to a region of the upper half, in one aspect of an embodiment to a region of the upper third, and in another aspect of an embodiment to a region of the upper quarter of the distillation unit. In another embodiment, a separating agent line can lead from the separation device into the upper region of the distillation unit. In another embodiment, a quenching agent line can lead from the separation device into an upper region, in one aspect of an embodiment into a region of the upper half, and in another aspect of an embodiment into a region of the upper third, and in a further aspect of an embodiment into a region of the upper quarter of the quenching unit. In a further embodiment, the quenching agent line can be interrupted by at least one purification device, in one aspect of an embodiment a crystallization device, in another aspect of an embodiment a suspension crystallization device comprising a crystal suspension generator and a washing column or a layer crystallizer. This purification device serves to separate off the (meth)acrylic acid still remaining in the quenched phase and to make the process according to the invention more economical.

In another embodiment of the present invention, the separation device can be a settling tank. In an aspect of an embodiment the separation device can be constructed and equipped such that the top product originating from the distillation unit is exposed to less movement in the separation device than in the distillation unit, in which a mixing operation is to be observed, in contrast to the separation device. Due to the dwell time in the separation device, a phase separation can be observed, at least one phase having a high content of separating agent and at least one aqueous phase having a low content of separating agent being formed.

Furthermore, in one embodiment of the device according to the invention a purification device follows the separation device. This serves in each case for purification of the phase of higher content of separating agent.

The present invention also relates to the process described above for the preparation of (meth)acrylic acid, in which the device described above is employed.

The present invention furthermore relates to a process for the preparation of a polymer, wherein a (meth)acrylic acid produced by the process described above is employed. In this context, the (meth)acrylic acid produced by the process according to the present invention, which, in one aspect of an embodiment can be the acrylic acid, is polymerized in the presence of free radical initiators and optionally crosslinking agents, in a process of solution polymerization, suspension polymerization or emulsion polymerization, optionally in the presence of further monomers which can be copolymerized with the (meth)acrylic acid produced by the process of the present invention and in the presence of crosslinking agents. The (meth)acrylic acid can be at least partly neutralized before, during or after the polymerization. In connection with the further monomers and crosslinking agents employed in the polymerization of the (meth)acrylic acid produced by the process according to the invention, reference is made to DE 101 61 495 A1, and there in particular to the monomers and crosslinking agents which are mentioned in this publication as monomers (a2) and as crosslinking agents (a3), DE 101 61 495 A1 herein specifically incorporated by reference.

In connection with the process according to the invention for the preparation of a polymer, the present invention also relates to a device for the preparation of such a polymer, which comprises a device as described above, followed by a polymerization device.

The present invention also relates to a process for the preparation of a polymer which is at least partly based on (meth)acrylic acid, wherein the device described above is employed for the preparation of this polymer.

The present invention also relates to fibers, films, foams and composites at least partly based on a (meth)acrylic acid produced by processes according to the invention for the preparation of (meth)acrylic acid or containing the polymer according to the invention described above.

The present invention also relates to the use of a (meth)acrylic acid produced by the process according to the invention for the preparation of (meth)acrylic acid or of a polymer produced by the process of the present invention for the preparation of a polymer in or for the production of fibers, films, foams and composites.

The invention will now be explained in more detail with the aid of non-limiting drawings and examples:

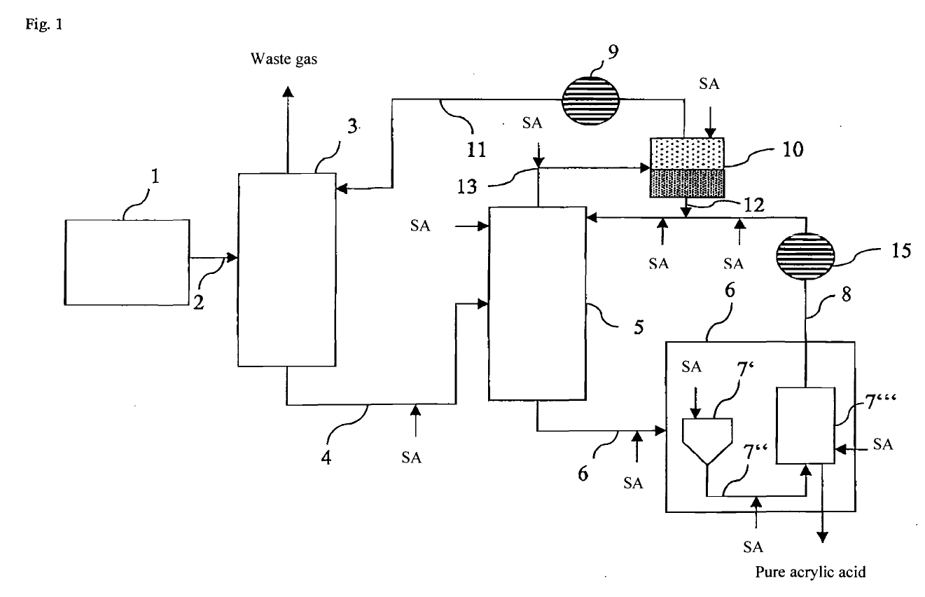

FIG. 1 shows the process according to the invention for the preparation of (meth)acrylic acid.

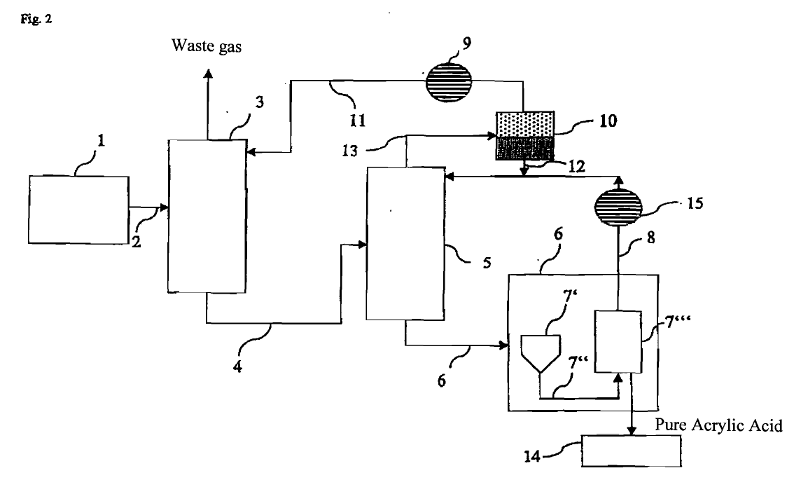

FIG. 2 shows the process according to the invention for the preparation of a polymer.

According to FIG. 1, the gaseous reaction product obtained in a reactor unit 1, which in the case of the oxidative gas phase oxidation of propylene comprises chiefly acrylic acid, water vapor, nitrogen, oxygen and by-products, such as, for example, maleic anhydride, is led via feed line 2 into the quenching unit 3. There, by bringing into contact with a liquid, such as, for example, water, the gaseous reaction product is absorbed in the liquid, in the case where water is used as the absorption liquid an aqueous acrylic acid solution which also contains, in addition to (meth)acrylic acid and the absorption agent, the by-products obtained in the gas phase oxidation being obtained. This aqueous (meth)acrylic acid solution is passed via feed 4 into the distillation unit 5, in which a separation of the (meth)acrylic acid from the absorption liquid (water), which can be in the presence of toluene as a separating agent, takes place. The single-phase bottom product which can be obtained in the distillation unit 5 and contains (meth)acrylic acid, the separating agent (SA, which can be toluene), a portion of the by-products obtained in the gas phase oxidation and small amounts of water, is fed by means of the feed 6 to the crystallization unit 7 comprising a crystallizer 7′, which can be a layer crystallizer or a suspension crystallization unit which comprises, in addition to a crystal suspension generator 7′, which can be a scraped film cooler, a separating device 7′″, which can be a washing column as the separating device 7′″ and a feed line 7″ for the crystal suspension into the separating device 7′″. In the separating device 7′″, in which the (meth)acrylic acid crystals are separated off, a mother liquor is retained, which can be at least partly recycled via the feed 8 into an upper region, in one aspect of an embodiment into a region of the upper half, in another aspect of an embodiment into a region of the upper third, and in a further aspect of an embodiment into a region of the upper quarter of the distillation unit 5 (in the case of a layer crystallizer, the mother liquor separated off is obtained directly in the crystallizer 7′). The top product obtained in the distillation unit 5 can be passed via feed line 13 into a separating device 10 in which the composition obtained as the top product in the distillation unit 5 undergoes a phase separation into two phases, one of which has a high content of separating agent and the other of which has a low content of separating agent. The phase having a low content of separating agent, which chiefly contains water, is then recycled via feed line 11 into the quenching unit 3. In a particular embodiment of the process according to the invention in this context (meth)acrylic acid still contained in the composition led in feed line 11 can be separated off from the composition by means of a purification device 9, in one embodiment by means of a crystallization unit. The phase having a high content of separating agent is recycled via feed line 12 into the distillation unit 5. It is furthermore preferable according to the invention for the composition led in feed line 8 (mother liquor separated off during the crystallization) to be freed from impurities by means of a separation unit 15 before being recycled.

In the case of a continuously operated purification process, feeding of fresh separating agent (SA) can take place
a) into the feed 4 via which the quenched phase is led into the distillation unit 5,
b) into the distillation unit 5,
c) into the feed 6 with which the bottom product obtained in the distillation unit 5 is led into the crystallization unit 7,
d) into the crystallizer 7′ of the crystallization unit,
e) into the feed 7″ with which, in the case where a suspension crystallizer is employed, the crystal suspension is led into the separating device 7′″,
f) into the separating device 7′″,
g) into the feed 8 with which the mother liquor obtained in the separating device 7′″ is recycled into the distillation unit 5,
h) into the separation device 10,
i) into the feed 12 in which the phase obtained in the separation device 10 having a high content of separating agent is recycled into the distillation unit 5,
j) into the feed 13 with which the top product of the distillation unit 5 is passed into the separation device 10.

Feeding in at several of the abovementioned points of the purification process is also possible.

According to FIG. 2, the acrylic acid obtainable by the process according to the invention is led into a polymerization device 14, in which a polymerization of the acrylic acid takes place, preferably in aqueous solution, in the presence of a crosslinking agent and optionally further comonomers, to form a water-absorbent polymer.

### EXAMPLE 1

In a large-scale industrial plant for the production of acrylic acid by means of gas phase oxidation of propylene, absorption of the resulting gas mixture in water to give an aqueous acrylic acid solution (quenched phase) and subsequent distillation of the quenched phase in the presence of toluene as a separating agent, the “Bottom product” shown in the following table was removed from the bottom of the distillation column following the quenching unit and fed to a cooling disc crystallizer from Gouda B.V. (Netherlands). The crystal suspension obtained by means of the cooling disc crystallizer was fed to a washing column which is described in DE 102 11 686 A1. The “Product” shown in the following table was obtained in this washing column, a separating agent phase with 56 wt. % of toluene moreover being retained as the mother liquor (all values in wt. %).

| | Bottom product | Product | Mother liquor |
|---|---|---|---|
| Acrylic acid | 86.5 | 99.477 | 21 |
| Toluene | 9.3 | 0.047 | 56 |
| MA¹⁾ | 1.0 | 0.00 | 6 |
| Acetic acid | 3.0 | 0.497 | 16 |
| Water | 0.2 | 0.021 | 1 |

¹⁾Maleic anhydride

### EXAMPLE 2

In a large-scale industrial plant for the production of acrylic acid by means of gas phase oxidation of propylene, absorption of the resulting gas mixture in water to give an aqueous acrylic acid solution (quenched phase) and subsequent distillation of the quenched phase in the presence of toluene as a separating agent, the “Bottom product”, the composition of which is given in the following table, was removed from the bottom of the distillation column following the quenching unit.

This bottom product was cooled to 0° C. in a laboratory scraped film cooler, the first crystals already forming at 6° C. The crystal suspension obtained in this way was spun to dryness in a CEPA drum centrifuge. The composition of the acrylic acid crystals obtained in this way and of the mother liquor is likewise given in the following table.

A portion of the acrylic acid crystals obtained was melted and used for washing the remaining crystals obtained after the centrifugation. The crystals washed with the molten acrylic acid were centrifuged to dryness again. The composition of the washed acrylic acid crystals obtained in this way is likewise given in the following table.

The mother liquor obtained after the first centrifugation step was introduced into the scraped film cooler again and was cooled again until a crystal suspension was obtained. This crystal suspension was spun to dryness in the centrifuge, acrylic acid crystals and mother liquor again being obtained. Some of the acrylic acid was melted and used for washing the remaining acrylic acid crystals, while the mother liquor was once again returned to the scraped film cooler. This procedure was repeated a total of another four times. The compositions of the acrylic acid crystals finally obtained, the washed acrylic acid crystals finally obtained and the mother liquor finally obtained are likewise given in the following table (all values in wt. %).

The overall yield of crystallized acrylic acid was 90° C. at a final temperature of −25° C.

| | BP¹⁾ | UAC(1)²⁾ | WAC(1)³⁾ | ML(1)⁴⁾ | UAC(1)⁵⁾ | WAC(6)⁶⁾ | ML(6)⁷⁾ |
|---|---|---|---|---|---|---|---|
| Water | 0.026 | 0.113 | 0.171 | 0.290 | 0.378 | 0.201 | 1.424 |
| Hydroquinone | 0.030 | 0.004 | <0.002 | 0.038 | 0.021 | <0.002 | 0.119 |
| Phenothiazine | 0.033 | 0.001 | <0.002 | 0.055 | 0.028 | <0.002 | 0.012 |
| Acrylic acid | 85.2 | 97.469 | 98.611 | 77.938 | 85.836 | 95.509 | 31.542 |
| Acetic acid | 2.53 | 0.966 | 0.673 | 3.510 | 4.454 | 2.858 | 8.747 |
| Propionic acid | 0.027 | 0.012 | 0.010 | 0.036 | 0.046 | 0.031 | 0.096 |
| Dimeric | 1.273 | 0.123 | 0.050 | 2.042 | 1.162 | 0.166 | 7.424 |
| acrylic acid | | | | | | | |
| Maleic | 0.102 | 0.014 | 0.014 | 0.180 | 0.530 | 0.233 | 0.190 |
| anhydride | | | | | | | |
| Furfural | 0.014 | 0.002 | 0.001 | 0.022 | 0.013 | 0.001 | 0.0748 |
| Benzaldehyde | 0.020 | 0.002 | 0.001 | 0.030 | 0.017 | 0.002 | 0.118 |
| Acrolein | 0.020 | <0.0001 | <0.0001 | 0.003 | 0.002 | <0.0001 | 0.009 |
| Protoanemonin | 0.012 | 0.001 | <0.0001 | 0.018 | 0.011 | <0.0001 | 0.066 |
| Toluene | 10.5 | 1.288 | 0.469 | 15.557 | 7.281 | 0.943 | 49.167 |
| Remainder | 0.176 | 0.005 | <0.002 | 0.281 | 0.105 | 0.057 | 1.022 |

¹⁾bottom product;
²⁾unwashed acrylic acid crystals after the first crystallization;
³⁾washed acrylic acid crystals after the first crystallization;
⁴⁾mother liquor after the first crystallization;
⁵⁾unwashed acrylic acid crystals after the sixth crystallization;
⁶⁾washed acrylic acid crystals after the sixth crystallization;
⁷⁾mother liquor after the sixth crystallization

### LIST OF REFERENCE SIGNS

1 Reactor unit
2 Feed for the reaction gases into the quenching unit
3 Quenching unit
4 Feed for acrylic acid absorbed in the quenching unit into the distillation unit 3
5 Distillation unit
6 Feed for the bottom product of the distillation unit into a crystallization unit 7 comprising the crystallizer′, the feed 7″ and, in the case of a suspension crystallizer, the washing column 7′″
7 Crystallization unit, which can be a suspension crystallization unit or layer crystallizer
7′ Crystallizer
7″ Feed for crystal suspension into the separating device 7′″, which can go into the washing column
7″″ Separating device, which can be a washing column
8 Feed for mother liquor into the top of the distillation unit 4
9 Optionally purification device
10 Separation device
11 Feed line for composition from the separation device having a low content of separating agent and based chiefly on water into the quenching unit
12 Recycling of phase separated off in the separation device 10 having a high content of separating agent into the distillation unit 4
13 Feed line for the top product of the distillation unit 4 into the separation device 10
14 Polymerization unit
15 Separating unit

1-25. (canceled)

26. A process for the preparation of (meth)acrylic acid, comprising the steps of:
(a) bringing into contact a product gas containing (meth)acrylic acid and obtained from a gas phase oxidation with an aqueous phase in a quenching unit to produce an aqueous quenched phase,
(b) subjecting the quenched phase to a distillation in a distillation unit to produce a bottom product and a top product,
(c) subjecting at least the bottom product to a crystallization in a crystallization unit to produce pure (meth)acrylic acid, and
(d) adding an organic separating agent after step (a) in the quenching unit and before or during step (c) in the crystallization unit.

27. The process according to claim 1, wherein the quenched phase comprises:
Q1 in the range of from about 45 to about 85 wt. % of (meth)acrylic acid,
Q2 at least about 14.9 wt. % of water, and
Q3 at least about 0.1 wt. % of impurities other than Q1 and Q2,
wherein the amounts stated in Q1 to Q3 are based on the total weight of the quenched phase, and wherein the sum of the wt. % stated for the quenched phase components Q1 to Q3 is 100.

28. The process according to claim 26, wherein the bottom product is a single phase.

29. The process according to claim 26, wherein the bottom product comprises:
S1 at least about 80 wt. % of (meth)acrylic acid,
S2 at most about 5 wt. % of water,
S3 at least about 1 wt. % of separating agent, and
S4 at least about 0.1 wt. % of impurities other than S1 to S4
wherein the amounts stated in S1 to S4 are based on the total weight of the bottom product and the sum of the wt. % stated for the bottom product components S1 to S4 is 100.

30. The process according to claim 26, wherein the pure (meth)acrylic acid comprises at least about 97.5 wt. % of (meth)acrylic acid.

31. The process according to claim 26, further comprising the steps of:
retaining the separating agent in step (c) in the crystallization unit, and
at least partly recycling the retained separating agent by adding again the retained separating agent after step (a) in the quenching unit and before or during step (c) in the crystallization unit.

32. The process according to claim 26, further comprising the step of at least partly separating the top product into separating agent and aqueous phase.

33. The process according to claim 32, further comprising the step of at least partly recycling the aqueous phase into the quenching unit.

34. The process according to claim 26, wherein the separating agent is an aromatic comprising at least one alkyl group.

35. The process according to claim 34, wherein the aromatic comprising at least one alkyl group is chosen from the group of toluene, o-xylene, m-xylene, p-xylene, mesitylene, 1,2,4-trimethylbenzene, ethylbenzene or p-cumene or mixtures of at least two of these.

36. A device for the preparation of (meth)acrylic acid comprising:
a reactor unit,
a quenching unit,
a distillation unit, and
a first crystallization unit comprising a crystallizer,
wherein the forgoing units are connected to one another in series by fluid-carrying lines.

37. The device according to claim 36 wherein the first crystallization unit is a suspension crystallization unit comprising a crystallizer and a separating device.

38. The device according to claim 37 wherein the separating device is a washing column.

39. The device according to claim 36, wherein the crystallizer of the first crystallization unit is connected to a region of the lower half of the distillation unit.

40. The device according to claim 36, wherein a separating agent feed from the first crystallization unit opens into a region of the upper half of the distillation unit.

41. The device according to claim 36, wherein a separator is connected to an upper region of the distillation unit.

42. The device according to claim 41, wherein a separating agent line leads from the separator into the upper region of the distillation unit.

43. The device according to claim 41, wherein a quenching agent line leads from the separator into an upper region of the quenching unit.

44. The device according to claim 41, wherein the separator is a settling tank.

45. The device according to claim 41, wherein a purification device follows the separator.

46. The device according to claim 45 wherein the purification device is a second crystallization unit.

47. A process for the preparation of a polymer, wherein a (meth)acrylic acid obtainable by a process according to claim 26 is polymerized.

48. A device for the preparation of a polymer, comprising a device according to claim 36 followed by a polymerization device.

49. A process for the preparation of a polymer at least partly based on (meth)acrylic acid, wherein a device according to claim 48 is employed.

50. Fibers, films, foams and composites, at least partly based on a (meth)acrylic acid obtainable by a process according to claim 26.

51. Use of a (meth)acrylic acid obtainable by a process according to claim 26 in or for the production of fibers, films, foams and composites.
